(19) United States
(12) Patent Application Publication
(10) Pub. No.: US 2021/0063294 A1
(43) Pub. Date: Mar. 4, 2021

(54) IN-LINE CONICAL VISCOMETER USING SHEAR STRESS SENSORS

(71) Applicant: Halliburton Energy Services, Inc., Houston, TX (US)

(72) Inventors: Xiangnan Ye, Cypress, TX (US); Dale E. Jamison, Atascocita, TX (US)

(73) Assignee: Halliburton Energy Services, Inc., Houston, TX (US)

(21) Appl. No.: 16/559,548

(22) Filed: Sep. 3, 2019

(51) Int. Cl. G01N 11/04; G01N 33/28; E21B 47/00; E21B 21/06

(52) U.S. Cl. CPC G01N 11/04; E21B 21/062; E21B 47/0006; G01N 33/2823

(57) ABSTRACT

A method of measuring a fluid's viscosity may include: flowing the fluid through a conduit wherein the conduit comprises shear stress sensors operable to measure shear stress on a wall of the conduit; measuring shear stress using the shear stress sensors; and calculating a viscosity of the fluid based at least in part on the measured shear stress.

### BACKGROUND

During the drilling of a wellbore into a subterranean formation, a drilling fluid, also referred to as a drilling mud, may be continuously circulated from the surface down to the bottom of the wellbore being drilled and back to the surface again. The drilling fluid serves several functions, one of them being to transport wellbore cuttings up to the surface where they are separated from the drilling fluid. Another function of the drilling fluid is to provide hydrostatic pressure on the walls of the drilled wellbore so as to prevent wellbore collapse and the resulting influx of gas or liquid from the formations being drilled.

Drilling fluids often include a plurality of particles that impart properties such as viscosity, density, and capabilities such as wellbore strengthening to the drilling fluid. Drilling fluid density is controlled such that the drilling fluid provides enough hydrostatic pressure to prevent invasion of formation fluids into the wellbore while not exceeding the fracture gradient of the formation thereby preventing fracturing of the formation. Weighting agents and viscosifiers can be used to produce drilling fluids with a desired viscosity, which affects the pumpability and equivalent circulating density (“ECD”) of the drilling fluid. The equivalent circulating density is the dynamic density exerted by the drilling fluid on the formation. As the drilling fluid is pumped through a drill string and out a drill bit, contact is made between the drilling, fluid and the wellbore walls as drilling, fluid flows upwards to the surface. This contact creates drag as a result of friction between the flowing drilling fluid and the wellbore walls and the drilling fluid loses some of the pressure supplied by the pump in other to overcome this frictional drag due. This pressure loss is absorbed by the wellbore walls so the equivalent circulating density is the sum of the pressure loss which may be converted to density and the original mud density of the drilling mud under static conditions.

During drilling operations, the ECD is often carefully monitored and controlled relative to the fracture gradient of the subterranean formation. Typically, the ECD during drilling is close to the fracture gradient without exceeding it. When the ECD exceeds the fracture gradient, a fracture may form in the subterranean formation and drilling fluid may be lost into the subterranean formation, often referred to as lost circulation, or formation fluids may rush into the wellbore causing a kick. The drilling fluid in the wellbore always exerts hydrostatic pressure on the wellbore walls, where the magnitude of the hydrostatic pressure is function of the drilling fluid density and vertical depth. The additional pressure felt by the formation or dynamic density referred to as ECD is a function of the viscosity of the drilling, fluid.

During drilling of a wellbore, the drill bit cuts into the formation causing the formation to break up and form pieces referred to as drill cuttings. These drill cuttings affect the viscosity of the drilling fluid, and therefore the ECD. The drill cuttings may be normally removed by size exclusion techniques such as filtering and gravity exclusion such as by cyclone. However, as the wellbore is drilled, the drill cuttings may be crushed to fine particles that do not readily separate by size exclusion or gravity methods. These difficult to remove solids may be referred to as low gravity solids and may affect the viscosity. Additives that modulate viscosity and other fluid parameters may be added to the drilling fluid to ensure that the ECD does not exceed safe limits for the formation.

### BRIEF DESCRIPTION OF THE DRAWINGS

These drawings illustrate certain aspects of some of the embodiments of the present disclosure and should not be used to limit or define the disclosure.

FIG. 1 is a schematic illustration of wall shear stress sensors within a tube.

FIG. 2 is a schematic illustration of shear stress sensors within a varying-diameter tube.

FIG. 3 is a schematic illustration of a drilling system that includes an in-line conical viscometer system according to at least some embodiments described herein.

FIG. 4 illustrates a block diagram of a drilling fluid monitoring and handling system according to at least some embodiments described herein.

### DETAILED DESCRIPTION

The embodiments described herein may relate to subterranean operations and, more particularly, more relate to in-line viscosity measurement systems and methods for measuring the viscosity of a fluid in a flow path using shear stress sensors. Such methods and apparatuses may be useful when integrated with drilling operations and systems for in-line measurement of drilling fluid viscosity

Present rheometer/viscometer designs for oilfield use usually involve measuring strain and torque of one or more moving parts exerted by a fluid. Some rheometer/viscometer designs may include parallel plate, cone and plate, and coaxial cylinder geometries, for example. Present viscometer designs may have certain limitations which make designing an in-line viscometer challenging. For example, material limitations of viscometer components such as seals that are suitable for high temperature and high pressure testing as well as resolution limitations generally preclude the use of traditional geometries in in line viscometers. While there are pipe flow rheometers available, pipe flow rheometers are generally limited to viscosity measurements of Newtonian fluids. Viscoelasticity and normal stress are typically not measurable with a pipe flow rheometer. Recently, oscillatory squeeze flow measurements have been used to determine the viscosity of a fluid between either two parallel plates or two coaxial surfaces. However, seal materials and resolution of strain and torque sensors are still a challenging to design around.

The present disclosure may relate to an in-line viscometer that includes shear stress sensors. The shear stress sensors may be disposed in a flow path and measure the shear stress exerted by a fluid flowing through the flow path which may then be used to determine fluid viscosity. The shear stress sensors may be any shear stress sensor capable of detecting shear stress and outputting a signal corresponding to the measured shear stress. In some examples, the shear stress sensors may be a micro-electro-mechanical system (MEMS) type sensor. Once the viscosity of the drilling fluid is known a determination may be made if any additives need to be added to keep the ECD of the drilling fluid within a specification.

The drilling fluid may include hydrocarbon-based drilling fluids, which may include a hydrocarbon liquid as the base fluid, which may be synthetic or oil-based. The drilling fluid may include an invert emulsion, which may include an external phase and an internal phase. The external phase may include a hydrocarbon liquid. The external phase can include dissolved materials or undissolved solids. Any suitable hydrocarbon liquid may be used in the external phase, including, but not limited to, a fractional distillate of crude oil; a fatty derivative of an acid, an ester, an ether, an alcohol, an amine, an amide, or an imide; a saturated hydrocarbon; an unsaturated hydrocarbon; a branched hydrocarbon; a cyclic hydrocarbon; and any combination thereof. Crude oil can be separated into fractional distillates based on the boiling point of the fractions in the crude oil. An example of a suitable fractional distillate of crude oil is diesel oil. The saturated hydrocarbon can be an alkane or paraffin. For example, the saturated hydrocarbon may be an isoalkane, a linear alkane, or a cyclic alkane. Examples of suitable saturated hydrocarbons may include a combination of an isoalkene and an n-alkane or a mineral oil blend that includes alkanes and cyclic alkanes. The unsaturated hydrocarbon may include an alkene, alkyne, or aromatic. The alkene may include an isoalkene, linear alkene, or cyclic alkene. The linear alkene may include a linear alpha olefin or an internal olefin. The hydrocarbon liquid may be present in the drilling fluid in an any suitable amount, including an amount of about 1 wt. % to about 90 wt. % based on a total weight of the drilling fluid. For example, the hydrocarbon liquid may be present in the drilling fluid in an amount of about 10 wt. %, about 20 wt. %, about 30 wt. %, about 40 wt. %, about 50 wt. %, about 60 wt. %, about 70 wt. %, about 80 wt. %, or about 90 wt. %, based on a total weight of the drilling fluid.

The internal phase may include an aqueous liquid. The aqueous liquid may be from any source provided that it does not contain an excess of compounds that may undesirably affect other components in the drilling fluids. For example, a drilling fluid may include fresh water or salt water. Salt water generally may include one or more dissolved salts therein and may be saturated or unsaturated as desired for a particular application. Seawater or brines may be suitable for use in some examples. The aqueous liquid may be present in the drilling fluid in an any suitable amount, including an amount of about 1 wt. % to about 90 wt. % based on a total weight of the drilling fluid. For example, the aqueous liquid may be present in the drilling fluid in an amount of about 10 wt. %, about 20 wt. %, about 30 wt. %, about 40 wt. %, about 50 wt. %, about 60 wt. %, about 70 wt. %, about 80 wt. %, or about 90 wt. %, based on a total weight of the drilling fluid.

As previously described, one or more dissolved salts may also be present in the aqueous liquid. Where used, the dissolved salt may be included in the aqueous liquid for any purpose, including, but not limited to, densifying a drilling fluid including water to a chosen density. A mixture of one or more dissolved salts and water may be used in some instances. The amount of salt that should be added may be the amount needed to provide a desired density. One or more salts may be added to the water to provide a brine that includes the dissolved salt and the water. Suitable dissolved salts may include monovalent (group I) and divalent salts (group II). Mixtures of monovalent, divalent, and trivalent salts may also be used. Suitable salts may include, but are not limited to, sodium chloride, calcium chloride, sodium bromide, potassium bromide, potassium chloride, potassium formate, cesium formate, lithium chloride, lithium bromide sodium formate, lithium formate, ammonium chloride, organic cation salts such as tetramethyl ammonium chloride, choline chloride, and mixtures thereof among others. The salt may be provided in any amount or concentration such as unsaturated, saturated, supersaturated, and saturated with additional solids. For example, the salt may be provided in an amount of about 1 wt. % to about 40 wt. % based on a total weight of the aqueous liquid. Alternatively, the salt may be present in the drilling fluid in an amount of about 1 wt. %, about 10 wt. %, about 20 wt. %, about 30 wt. %, or about 40 wt. % based on a total weight of the drilling fluid.

The drilling fluids may include an emulsifying surfactant. Some examples of emulsifying surfactants may include, without limitation, fatty amines, ethoxylated nonylphenols, fatty acids, fatty acid esters, and combinations thereof. Emulsifying surfactants may be present in any amount suitable for a particular application. In some examples, without limitation, the emulsifying surfactant may be present in the drilling fluid in an amount of about 0.5 wt. % to about 10 wt. % based on a total weight of the drilling fluid. Specific amounts of the emulsifying surfactant may include, but are not limited to about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % based on a total weight of the drilling fluid.

The drilling fluids may include a clay. Any of a variety of different clays may be included in the drilling fluids. Suitable clays may include, but are not limited to, sepiolite, attapulgite, calcium bentonite, sodium bentonite, calcium montromillonite, organoclays, and combinations thereof. Organoclays are organically modified phyllosilicate formed by exchanging interlayer canons for alkylamonium or phosphonium ions. The clay may be present in any suitable amount for a particular application, including, but not limited to, an amount ranging from about 1 wt. % to about 50 wt. % based on a total weight of the drilling fluid. For example, the clay may be present in an amount of about 1 wt %, about 10 wt. %, about 20 wt. %, about 30% wt. %, about 40 wt. %, or about 50 wt. % based on a total weight of the drilling fluid.

A wide variety of additional additives may be included in the drilling fluids as desired for a particular application. Suitable additives may include, but are not limited to, viscosifiers, shale stabilizers, wetting agents, and weighting agents, among others. Suitable viscosifiers may include, but are not limited to, water soluble starches and modified versions thereof, water-soluble polysaccharides and modified versions thereof, water soluble celluloses and modified versions thereof, water soluble polyacrylamides and copolymers thereof, biopolymers, and combinations thereof.

The drilling fluid generally should have a density suitable for a particular application. By way of example, the drilling fluid may have a density of about 7 pounds per gallon (“lb/gal”) (838.8 kg/m³) to about 20 lb/gal (2397 kg/m³). In certain embodiments, the drilling fluid may have a density of about 8 lb/gal (958.6 kg/m³) to about 12 lb/gal (1438 kg/m³).

When drilling a wellbore, the drilling fluid may be continuously circulated from the well surface down to the bottom of the wellbore being drilled and back to the well surface again. The composition of the drilling fluid may change during the course of the drilling fluid due to a number of factors, including, but not limited to, the loss of drilling fluid additives in the wellbore and the addition of drill solids into the drilling fluid. To maintain adequate properties of the drilling fluid, the drilling fluid may be monitored to determine its viscosity at the surface while it is being circulated.

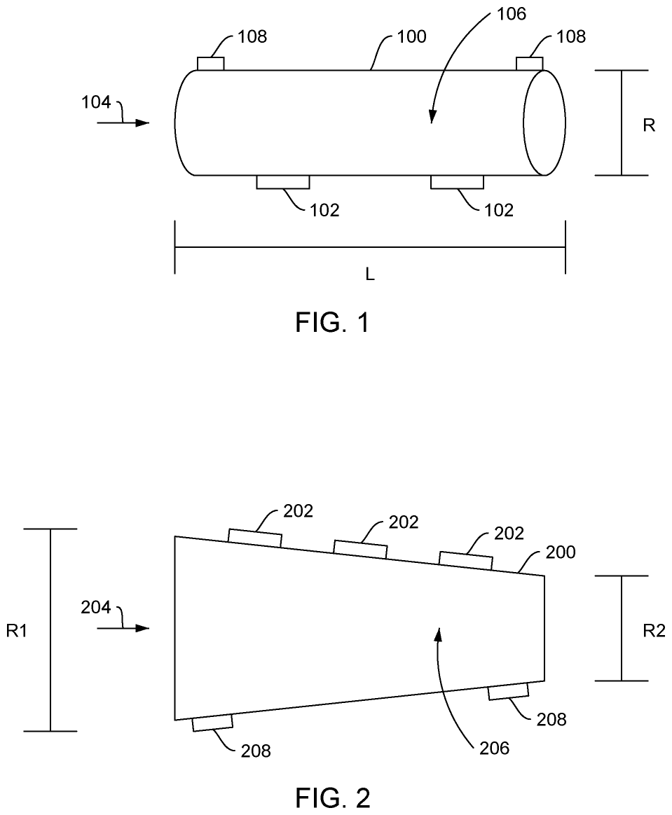

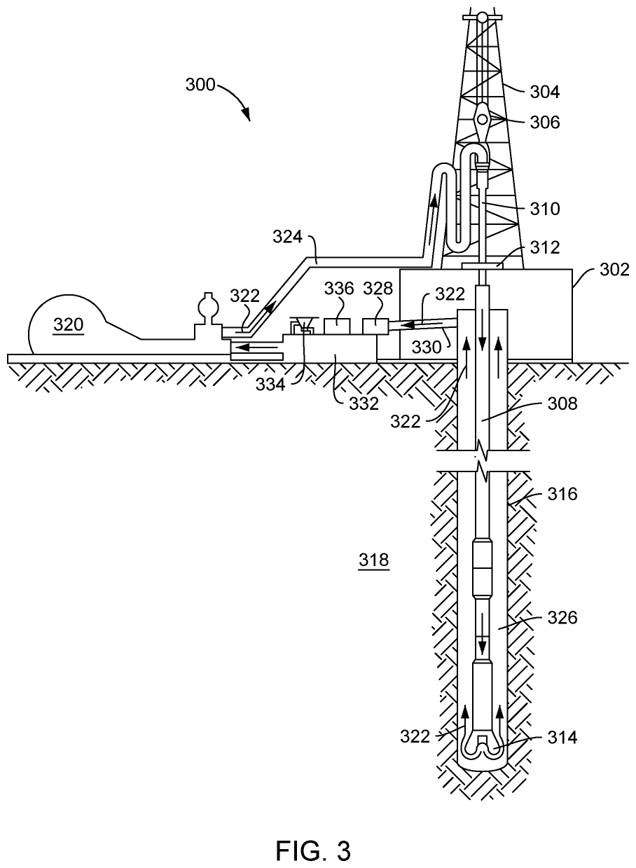

FIG. 1 illustrates a tubular in-line viscometer. The tubular in-line viscometer may have a constant diameter R across a length L. A constant diameter means the diameter does not vary more than 5% from a first position along length L to a second position along length L. In FIG. 1, tube 100 is illustrated with shear sensors 102 disposed on a wall of tube 100. Shear sensors 102 may be exposed to flow path 106 defined by an interior of tube 100. Fluid 104 may be introduced into tube 100 at a known flow rate, Q, and the shear sensors 102 may detect shear caused by the flow of fluid 104 through tube 100. The shear stress data generated by shear sensors 102 may be used to calculate rheology parameters according to the mathematical model developed below, for example. Pressure sensors 108 may be used to detect pressure drop across tube 100 in the direction of fluid flow.

Drilling fluid rheological behavior may be modeled using rheological models such as Herschel-Bulkley, Bingham plastic, and power law. In an example, a power law may be used as a model of the drilling fluid rheology as shown in Equation 1.

τ=K\dot{y}ⁿ (1)

where τ=shear stress, K=viscosity constant, \dot{y}=shear rate, and n=power law exponent

$$\dot{y} = - \frac{d v}{dr}$$

The shear rate \dot{y} within a tube, such as a viscometer, may be expressed as with v being the velocity and r being the radius of the tube. Given the wall shear stress of τ_{w}, the shear stress within the tube may be written as

$$τ = {τ}_{w} \frac{r}{R}$$

where R is the ammeter of the pipe and r is the distance from the center of the pipe. As such, equation 1 may be re-written as Equation 2.

$$- \frac{d v}{dr} = {( \frac{r {τ}_{w}}{R K} )}^{\frac{1}{n}} \qquad ( 2 )$$

Volumetric flow rate through the tube is shown in Equation 3 which when integrated by parts gives Equation 4.

$$\begin{matrix}d Q = 2 π rvdr & ( 3 ) \\ Q = 2 π {\int }_{0}^{R} r v d r = 2 π {\{ \frac{{τ}^{2} v}{2} \vert}_{0}^{R} + {\int }_{0}^{R} \frac{{r}^{2}}{2} ( - \frac{d v}{dr} ) d r \} & ( 4 )\end{matrix}$$

The no-slip boundary condition causes the first term in the brackets of Equation 4 to reduce to zero.

Equations 2 and 4 may be combined to form Equation 5.

$$Q = π {\int }_{0}^{R} {{r}^{2} ( \frac{r {τ}_{w}}{R K} )}^{1 / n} d r \qquad ( 5 )$$

Equation 5 may be integrated to yield Equation 6.

$$Q = \frac{n π}{3 n + 1} {( \frac{{τ}_{w}}{K} )}^{\frac{1}{n}} {R}^{3 n} \qquad ( 6 )$$

Equation 6 may be used to solve for the parameters n and K in Equation 1 which may then be used to determine the shear stress. The parameters n and K may be solved for by varying flow rate Q through the tube and measuring the resultant τ_{w} with the shear sensors disposed in the tube, for example.

Another fluid model that may be used to model a drilling fluid is a Herschel-Bulkley model. The Herschel-Bulkley model is illustrated in Equation 7 where τ₀ is the yield stress. The yield stress may be considered a model parameter and may be determined from measured Q and τ_{w} data.

τ=τ₀+K\dot{y}ⁿ (7)

Equation 7 may be integrated using the flow rate equation supplied above and the no slip boundary condition to yield Equation 8. In equation 8, the three different flow rates may be required to determine the model parameters τ₀, K, and n.

$$Q = {( \frac{{τ}_{w} - {τ}_{0}}{K} )}^{\frac{1}{n}} \{ \frac{π {R}^{3}}{3} + \frac{{τ}_{w} π R}{K} * \frac{n}{n + 1} \} + \frac{{τ}_{w} π R}{K} * \frac{n}{n + 1} ( \frac{{τ}_{w} - {τ}_{0}}{K} ) + \frac{π {R}^{3}}{3} {( \frac{{τ}_{0}}{K} )}^{\frac{1}{n}} \qquad ( 8 )$$

FIG. 2 illustrates an alternate embodiment of an in-line viscometer utilizing a tube 200 with a varying diameter geometry. In FIG. 2, fluid 204 may flow through flow path 206 defined by an interior of tube 200. Fluid 104 may be introduced into tube 200 at a known flow rate, Q, and the shear sensors 202 may detect shear caused by the flow of fluid 204 through tube 200. The shear stress data may be used to calculate rheology parameters. Pressure sensors 208 may be used to detect pressure drop across tube 200 in the direction of fluid flow. The radius of tube 200 may begin at a first diameter R1 and end at a second diameter R2 where R1≠R2. Tube 200 may be any geometry with two different diameters including, but not limited to, conical geometries and step change geometries, for example. A conical geometry may include a conical frustum geometry as illustrated in FIG. 2, for example.

The configuration of shear sensors 202 along a varying geometry tube depicted in FIG. 2 may be advantageous in that the viscosity profile may be determined by one flow rate. For a fluid model utilizing two parameters such as a power law model, the model parameters may be determined by using two shear sensors. For a fluid model utilizing three parameters such as a Herschel-Bulkley model, the model parameters may be determined using three shear sensors. For the geometry of FIG. 2, Equation 9 and Equation 10 may be utilized for power law models and Herschel-Bulkley models respectively. In Equations 9 and 10, τ_{wi} is the shear stress measured at radius Ri corresponding to a shear stress sensor disposed within tube 200 at radius Ri.

$$\begin{matrix} Q = \frac{n π}{3 n + 1} {( \frac{{τ}_{wi}}{K} )}^{\frac{1}{n}} {R}_{i}^{3 n} & ( 9 ) \\ Q = {( \frac{{τ}_{wi} - {τ}_{0}}{K} )}^{\frac{1}{n}} \{ \frac{π {R}^{3}}{3} + \frac{{τ}_{wi} π {R}_{i}}{K} * \frac{n}{n + 1} \} + \frac{{τ}_{wi} π {R}_{i}}{K} * \frac{n}{n + 1} ( \frac{{τ}_{wi} - {τ}_{0}}{K} ) + \frac{π {R}_{i}^{3}}{3} {( \frac{{τ}_{0}}{K} )}^{\frac{1}{n}} & ( 10 )\end{matrix}$$

Once the viscosity has been determined using any of the above-mentioned methods, a determination may be made that an additional amount of chemical additive may be required to be added to the drilling fluid to maintain the ECD of the drilling fluid within a specified limit. Some additives may include suspending aids such as chemical agents which increase the viscosity of the drilling fluid or base fluid to dilute the drilling fluid to decrease viscosity, for example. As the rheological properties of a drilling fluid may an indication of the ability of a drilling fluid to suspend solid particles, modulating viscosity using suspending aids and base fluid may ensure that the drilling fluid is able to suspend drill cuttings without the cutting dropping out of the drilling fluid and not exceeding the maximum ECD for a particular application.

As illustrated, the drilling assembly 300 may include a drilling platform 302 that supports a derrick 304 having a traveling block 306 for raising and lowering a drill string 308. The drill string 308 may include, but is not limited to, drill pipe and coiled tubing, as generally known to those skilled in the art. A kelly 310 supports the drill string 308 as it is lowered through a rotary table 312. A drill bit 314 is attached to the distal end of the drill string 308 and is driven either by a downhole motor and/or via rotation of the drill string 308 from the well surface. As the drill bit 314 rotates, it creates a wellbore 316 that penetrates various subterranean formations 318.

A pump 320 (e.g., a mud pump) circulates drilling fluid 322 through a feed pipe 324 and to the kelly 310, which conveys the drilling fluid 322 downhole through the interior of the drill string 308 and through one or more orifices in the drill bit 314. The drilling fluid 322 is then circulated back to the surface via an annulus 326 defined between the drill string 308 and the walls of the wellbore 316. At the surface, the recirculated or spent drilling fluid 322 exits the annulus 326 and may be conveyed to one or more fluid processing unit(s) 328 (e.g., shakers) via an interconnecting flow line 330. The one or more fluid processing unit(s) 328 may be useful in removing large drill cuttings that may interfere with the viscosity measurements described herein. After passing through the fluid processing unit(s) 328, a “cleaned” drilling fluid 322 is deposited into a nearby retention pit 332 (i.e., a mud pit). While illustrated as being arranged at the outlet of the wellbore 316 via the annulus 326, those skilled in the art will readily appreciate that the fluid processing unit(s) 328 may be arranged at any other location in the drilling assembly 300 to facilitate its proper function, without departing from the scope of the disclosure.

One or more additives (e.g., weighting agents) may be added to the drilling fluid 322 via a mixing hopper 334 communicably coupled to or otherwise in fluid communication with the retention pit 332. The mixing hopper 334 may include, but is not limited to, mixers and related mixing equipment known to those skilled in the art. In other embodiments, however, additives may be added to the drilling fluid 322 at any other location in the drilling assembly 300. In at least one embodiment, for example, there could be more than one retention pit 332, such as multiple retention pits 332 in series. Moreover, the retention pit 332 may be representative of one or more fluid storage facilities and/or units where additives may be stored, reconditioned, and/or regulated until added to the drilling fluid 322.

The drilling assembly 300 may include one or more in-line viscometer system 336 in fluid communication with the at least one retention pit 332. Samples of the drilling fluid in the retention pits 332 may be transported to the in-line viscometer system 336 to measure the viscosity of the drilling fluid 322. Further, based on the viscosity measurements, one or more additives may be added to the drilling fluid via the mixing hopper 334 to adjust the viscosity of the drilling fluid to a desired value.

While not specifically illustrated herein, the drilling assembly 300 may also include additional components, for example, shakers (e.g., shale shaker), centrifuges, hydrocyclones, separators (e.g., magnetic and electrical separators), desilters, desanders, filters (e.g., diatomaceous earth filters), heat exchangers, fluid reclamation equipment, sensors, gauges, pumps, compressors, conduits, pipelines, trucks, tubulars, pipes, pumps, compressors, motors, valves, floats, drill collars, mud motors, downhole motors, downhole pumps, MWD/LWD tools, tool seals, packers, roller cone bits, PDC bits, natural diamond bits, any hole openers, reamers, coring bits, and the like, and any communication components associated therewith (e.g., wirelines, telemetry components, etc.).

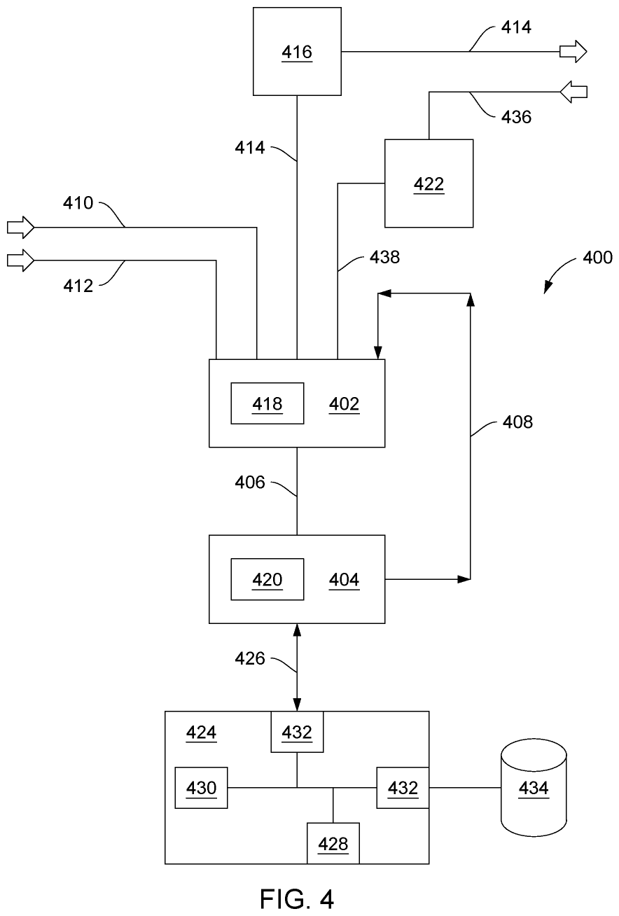

FIG. 4 illustrates a block diagram of a drilling fluid monitoring and handling system 400 for determining concentration of one or more components of drilling fluids. As illustrated, the fluid monitoring and handling system 400 may generally include a mud pit 402 and a fluid analysis system 404. A portion of the drilling fluid from the mud pit 402 may be fed via a mud pit line 406 to the fluid analysis system 404, which may be configured to perform rheology measurements using an in-line viscometer on the portion of the drilling fluid supplied thereto. The in-line viscometer may be any of the viscometers described herein such as those utilizing shear stress sensors. The fluid analysis system 404 may analyze the drilling fluid using any of the methods disclosed herein. After fluid analysis, the portion of the drilling fluid may be returned to mud pit 402 via a return line 408.

The mud pit 402 may be any vessel suitable for holding a drilling fluid. For example, the mud pit 402 may include a container such as a drum or tank, or a series of containers that may or may not be connected. The mud pit 402 may be supplied with the drilling fluid from an initial drilling fluid supply line 410 that provides an initial supply of drilling fluid to the mud pit 402. However, the initial supply of drilling fluid does not imply that the drilling fluid has not been recycled or circulated in a wellbore, but simply indicates that this supply is not presently being circulated or otherwise used in the wellbore.

Drilling fluid additives (e.g., emulsifying agents, clay, viscosifiers, etc.) may be added via a drilling fluid additive supply line 412 to the mud pit 402, based at least in part on the analysis provided by the fluid analysis system 404. For example, if measured viscosity is above a setpoint for a particular application, one or more chemical additives, base fluid, or both may be added via fluid additive supply line 412 such that the viscosity of the drilling fluid is reduced. Alternatively, if the viscosity is determined to be below a setpoint, one or more chemical additives, base fluid, or both may be added via fluid additive supply line 412 such that the viscosity of the drilling fluid is increased. Alternatively or additionally, the results of the analysis may be used to modify the manufacturing process of the drilling fluid. After the drilling fluid additives have been added to the drilling fluid, the drilling fluid may be retested using the fluid analysis system 404 to verify the drilling fluid was correctly formulated or the drilling fluid may be sent to the wellbore for use in drilling operations via a wellbore line 414 by way of mud pump 416.

The mud pit 402 may include a mixing system 418 to mix the contents of the mud pit 402 as well as any drilling fluid additives. For instance, the mixing system 418 may mix the drilling fluid in the mud pit 402 with drilling fluid from the initial drilling fluid supply line 410, drilling fluid from the return line 408, drilling fluid additives, additional non-aqueous fluids, aqueous fluids or combinations thereof. In general, the mixing system 418 may be configured to prevent solids within the drilling fluid from settling. The mixing system 418 may use any suitable mixing technique for mixing of the drilling fluid. For instance, the mixing system 418 may include a static mixer, dynamic mixer, or other suitable mixer. The mud pit 402 may further include suitable pumping equipment (not shown) t to pump the drilling fluid in the mud pit 402 to the fluid analysis system 404 via mud pit line 406.

The fluid analysis system 404 may analyze the portion of the drilling fluid in a continuous or non-continuous manner, as desired, and based on whether flow through fluid analysis system 404 is continuous or non-continuous. The fluid analysis system 404 may include one or more instruments 420 for measuring rheology of the drilling fluid such the in-line viscometers discussed herein. while applying an electric field to the drilling fluid. For example, the instruments 420 may include a rheometer as described herein as well as any combination of densometers, gel testing equipment, oil-to-water ratio testing equipment, water phase salinity equipment, pH testing equipment, for example.

Although the fluid analysis system 404 is shown at the mud pit 402, examples disclosed herein contemplate the placement of fluid analysis system 404 at any point in the fluid monitoring and handling system 400. For example, one or more instruments 420 of the fluid analysis system 404 may alternatively be placed in a fluid reconditioning system 422 (discussed below), the mud pit 402, as well as within the wellbore or in an exit conduit from the wellbore. As such, examples disclosed herein contemplate measuring the rheology using the disclosed in-line viscometers at any point in the drilling fluid handling process, so that the drilling fluid may be monitored and/or subsequently adjusted as desired.

The analysis performed by fluid analysis system 404 may be performed in collaboration with a computer system 424 communicably coupled thereto. As illustrated, the computer system 424 may be an external component of the fluid analysis system 404, however, the computer system 424 may alternatively include an internal component of the fluid analysis system 404, without departing from the scope of the disclosure. The computer system 424 may be connected to the fluid analysis system 404 via a communication link 426. The communication link 426 may include a direct (wired) connection, a private network, a virtual private network, a local area network, a WAN (e.g., an Internet-based communication system), a wireless communication system (e.g., a satellite communication system, telephones), any combination thereof, or any other suitable communication link.

The computer system 424 may be any suitable data processing system including, but not limited to, a computer, a handheld device, or any other suitable device. The computer system 424 may include a processor 428 and a non-transitory computer readable storage medium 430 communicatively coupled to the processor 428. The processor 428 may include one central processing unit or may be distributed across one or more processors in one or more locations. Examples of a non-transitory computer readable storage medium 430 include random-access memory (RAM) devices, read-only memory (ROM) devices, optical devices (e.g., CDs or DVDs), disk drives, and the like. The non-transitory computer readable storage medium 430 may store computer readable program code that may be executed by the processor 428 to process and analyze the measurement data generated by fluid analysis system 404, adjust the parameters of the fluid monitoring and handling system 400, and/or operate a part or whole of the fluid monitoring and handling system 400. Further, from the rheological measurements of the drilling fluid measured by the fluid analysis system 404 while an electric field is applied, the program code may be executed by the processor 428 to calculate the parameters of a viscosity model and determine viscosity of the measured drilling fluid.

The computer system 424 may further include one or more input/output (“I/O”) interface(s) 432 communicatively coupled to the processor 428. The I/O interface(s) 432 may be any suitable system for connecting the computer system 424 to a communication link, such as a direct connection, a private network, a virtual private network, a local area network, a wide area network (“WAN”), a wireless communication system, or combinations thereof; a storage device, such as storage 434; an external device, such as a keyboard, a monitor, a printer, a voice recognition device, or a mouse; or any other suitable system. The storage 434 may store data required by the fluid analysis system 804 for performing fluid analysis. The storage 434 may be or include compact disc drives, floppy drives, hard disks, flash memory, solid-state drives, and the like.

Data processing and analysis software native to the fluid analysis system 404 and/or installed on the computer system 424 may be used to analyze the data generated by fluid analysis system 404. This procedure may be automated such that the analysis happens without the need for operator input or control. Further, the operator may select from several previously input parameters or may be able to recall previously measured data. Any of the data may be transferred and/or stored on an external memory device (e.g., a USB drive), if desired.

With continued reference to FIG. 4, the drilling fluid may be delivered to a wellbore from mud pit 402 by way of mud pump 416 via wellbore line 414. The mud pump 416 may be any type of pump or pumping system useful for circulating a drilling fluid into a subterranean formation under a sufficient pressure. The drilling fluid that has been circulated within the wellbore may be returned to the mud pit 402 via a circulated drilling fluid return line 436 and provided to a fluid reconditioning system 422 to condition the circulated drilling fluid prior to returning it to the mud pit 402. The fluid reconditioning system 422 may be or include one or more of a shaker (e.g., shale shaker), a centrifuge, a hydrocyclone, a separator (including magnetic and electrical separators), a desilter, a desander, a separator, a filter (e.g., diatomaceous earth filters), a heat exchanger, and any fluid reclamation equipment. The fluid reconditioning system 422 may further include one or more sensors, gauges, pumps, compressors, and the like used to monitor, regulate, and/or recondition the drilling fluid and various additives added thereto. After the drilling fluid has been reconditioned, the drilling fluid may be returned to the mud pit 402 via the reconditioned fluid line.

The systems described herein may be useful in measuring the viscosity of a drilling fluid while drilling a wellbore penetrating a subterranean formation and may allow for changing the viscosity of the drilling fluid during such an operation. For example, after removing the larger drill cuttings (e.g., 1 mm or larger) with shakers, centrifuges, or the like, the viscosity of the drilling fluid may be measured. Then, the viscosity of the drilling fluid may be increased or decreased to meet the requirements of the drilling operation. For example, weighting agents, viscosifiers, or the like may be added to increase viscosity, while a breaker, additional base fluid, or the like may be added to decrease the viscosity.

Accordingly, the present disclosure may provide methods, systems, and apparatus that may relate to in-line viscosity measurement systems and methods for measuring the viscosity of a fluid in a flow path using shear stress sensors. The methods, systems, and apparatus may include any of the various features disclosed herein, including one or more of the following statements.

Statement 1. A method of measuring a fluid's viscosity comprising: flowing the fluid through a conduit wherein the conduit comprises shear stress sensors operable to measure shear stress on a wall of the conduit; measuring shear stress using the shear stress sensors; and calculating a viscosity of the fluid based at least in part on the measured shear stress.

Statement 2. The method of statement 1 wherein the shear stress sensors comprise a MEMS shear sensor.

Statement 3. The method of any of statements 1-2 wherein the step of calculating a viscosity comprises: calculating rheological model parameters from a flow rate of the fluid and measured shear stress at the flow rate; and calculating the viscosity based at least in part on a rheological model that corresponds to the calculated rheological model parameters.

Statement 4. The method of statement 3 wherein the rheological model is a power law model in the form of:

τ=K\dot{y}ⁿ

where τ=shear stress and the theological model parameters are K=viscosity constant, \dot{y}=shear rate, and n=power law exponent.

Statement 5. The method of statement 4 wherein the rheological model parameters are calculated based at least in part on the following equation:

$$Q = \frac{n π}{3 n + 1} {( \frac{{τ}_{w}}{K} )}^{\frac{1}{n}} {R}^{3 n}$$

where τ_{w} measured shear stress and R is radius.

Statement 6. The method of any of statements 1-5 wherein the conduit is a conical frustum with a variable diameter and wherein the rheological model parameters are calculated based at least in part on the following equation:

$$Q = \frac{n π}{3 n + 1} {( \frac{{τ}_{wi}}{K} )}^{\frac{1}{n}} {R}_{i}^{3 n}$$

where τ_{wi} is the shear stress measured at radius Ri.

Statement 7. The method of any of statements 1-3 wherein the rheological model is a Herschel-Bulkley model in the form of:

τ=τ₀+K\dot{y}ⁿ

where τ=shear stress, τ₀ is yield stress, and the rheological model parameters are K=viscosity constant, \dot{y}=shear rate, and n=power law exponent.

Statement 8. The method of any of statement 3 wherein the rheological model parameters are calculated based at least in part on the following equation:

$$Q = {( \frac{{τ}_{w} - {τ}_{0}}{K} )}^{\frac{1}{n}} \{ \frac{π {R}^{3}}{3} + \frac{{τ}_{w} π R}{K} * \frac{n}{n + 1} \} + \frac{{τ}_{w} π R}{K} * \frac{n}{n + 1} ( \frac{{τ}_{w} - {τ}_{0}}{K} ) + \frac{π {R}^{3}}{3} {( \frac{{τ}_{0}}{K} )}^{\frac{1}{n}}$$

where τ_{w} measured shear stress and R is radius.

Statement 9. The method of claim 3 wherein the rheological model parameters are calculated based at least in part on the following equation:

$$Q = {( \frac{{τ}_{wi} - {τ}_{0}}{K} )}^{\frac{1}{n}} \{ \frac{π {R}^{3}}{3} + \frac{{τ}_{wi} π {R}_{i}}{K} * \frac{n}{n + 1} \} + \frac{{τ}_{wi} π {R}_{i}}{K} * \frac{n}{n + 1} ( \frac{{τ}_{wi} - {τ}_{0}}{K} ) + \frac{π {R}_{i}^{3}}{3} {( \frac{{τ}_{0}}{K} )}^{\frac{1}{n}}$$

where τ_{wi} is the shear stress measured at radius Ri.

Statement 10. A method comprising: circulating a drilling fluid through a wellbore penetrating a subterranean formation while drilling the wellbore; flowing the fluid through a conduit wherein the conduit comprises shear stress sensors operable to measure shear stress on a wall of the conduit; measuring shear stress using the shear stress sensors; and calculating a viscosity of the fluid based at least in part on the measured shear stress.

Statement 11. The method of statement 10 wherein the shear stress sensors comprise a MEMS shear sensor.

Statement 12. The method of any of statements 10-11 wherein the step of calculating a viscosity comprises: calculating rheological model parameters from a flow rate of the drilling fluid and measured shear stress at the flow rate; and calculating the viscosity based at least in part on a rheological model that corresponds to the calculated rheological model parameters.

Statement 13. The method of any of statements 10-12 wherein the rheological model is a power law model in the form of:

τ=K\dot{y}ⁿ

where τ=shear stress and the rheological model parameters are K=viscosity constant, \dot{y}=shear rate, and n=power law exponent, and wherein the rheological model parameters are calculated based at least in part on the following equation:

$$Q = \frac{n π}{3 n + 1} {( \frac{{τ}_{wi}}{K} )}^{\frac{1}{n}} {R}_{i}^{3 n}$$

where τ_{wi} is the shear stress measured at radius Ri.

Statement 14. The method of any of statements 10-13 wherein the conduit has a conical frustum geometry, wherein the rheological model is a Herschel-Bulkley model in the form of:

τ=τ₀+K\dot{y}ⁿ

where τ=shear stress, τ₀ is yield stress, and the rheological model parameters are K=viscosity constant, \dot{y}=shear rate, and n=power law exponent, and wherein the rheological model parameters are calculated based at least in part on the following equation:

$$Q = {( \frac{{τ}_{wi} - {τ}_{0}}{K} )}^{\frac{1}{n}} \{ \frac{π {R}^{3}}{3} + \frac{{τ}_{wi} π {R}_{i}}{K} * \frac{n}{n + 1} \} + \frac{{τ}_{wi} π {R}_{i}}{K} * \frac{n}{n + 1} ( \frac{{τ}_{wi} - {τ}_{0}}{K} ) + \frac{π {R}_{i}^{3}}{3} {( \frac{{τ}_{0}}{K} )}^{\frac{1}{n}}$$

where τ_{wi} is the shear stress measured at radius Ri.

Statement 15. The method of any of statements 10-14 further comprising: comparing the viscosity of the drilling fluid to a setpoint viscosity; calculating an amount of a chemical additive to add to the drilling fluid to reach the setpoint viscosity; and adding a chemical additive to the drilling fluid based at least in part on the calculating.

Statement 16. The method of claim 15 wherein the chemical additive comprises at least one selected from the group consisting of a weighting agent, a viscosifier, a breaker, a base fluid, and combinations thereof.

Statement 17. A system comprising: a line fluidly connecting a mixing tank and a tubular extending into a wellbore with a pump disposed along the line between the mixing tank and the tubular; a conduit in fluid communication with the line between the mixing tank and the pump; and one or more shear stress sensors disposed within the conduit operable to measure shear stress on a wall of the conduit.

Statement 18. The system of statement 17 further comprising at least one chemical additive tank coupled to the mixing tank.

Statement 19. The system of statement 18 further comprising a control system operable to calculate a viscosity of a fluid within the conduit, compare the viscosity to a setpoint viscosity, calculate an amount of a chemical additive to add to the mixing tank such that the viscosity of the fluid is adjusted to a value closer to the setpoint viscosity.

Statement 20. The system of statement 19 wherein the control system is configured to calculate viscosity by calculating rheological model parameters from a flow rate of the fluid through the conduit and measured shear stress at the flow rate, and calculate the viscosity based at least in part on a rheological model that corresponds to the calculated rheological model parameters.

The exemplary spacer fluid disclosed herein may directly or indirectly affect one or more components or pieces of equipment associated with the preparation, delivery, recapture, recycling, and/or use of drilling fluids. For example, the drilling fluids (or components thereof) may directly or indirectly affect one or more mixers, related mixing equipment, mud pits, storage facilities or units, composition separators, heat exchangers, sensors, gauges, pumps, compressors, and the like used generate, store, monitor, regulate, and/or recondition the exemplary sugar cane ash and fluids containing the same. The disclosed viscometer (or components thereof) may also directly or indirectly affect any transport or delivery equipment used to convey the drilling fluid (or components thereof) to a well site or downhole such as, for example, any transport vessels, conduits, pipelines, trucks, tubulars, and/or pipes used to compositionally move the drilling fluid (or components thereof) from one location to another, any pumps, compressors, or motors (e.g., topside or downhole) used to drive the drilling fluid (or components thereof), into motion, any valves or related joints used to regulate the pressure or flow rate of the drilling fluid, and any sensors (i.e., pressure and temperature), gauges, and/or combinations thereof, and the like. The disclosed drilling fluid may also directly or indirectly affect the various downhole equipment and tools that may come into contact with the drilling fluid such as, but not limited to, wellbore casing, wellbore liner, completion string, insert strings, drill string, coiled tubing, slickline, wireline, drill pipe, drill collars, mud motors, downhole motors and/or pumps, cement pumps, surface-mounted motors and/or pumps, centralizers, turbolizers, scratchers, floats (e.g., shoes, collars, valves, etc.), logging tools and related telemetry equipment, actuators (e.g., electromechanical devices, hydromechanical devices, etc.), sliding sleeves, production sleeves, plugs, screens, filters, flow control devices (e.g., inflow control devices, autonomous inflow control devices, outflow control devices, etc.), couplings (e.g., electro-hydraulic wet connect, dry connect, inductive coupler, etc.), control lines (e.g., electrical, fiber optic, hydraulic, etc.), surveillance lines, drill bits and reamers, sensors or distributed sensors, downhole heat exchangers, valves and corresponding actuation devices, tool seals, packers, cement plugs, bridge plugs, and other wellbore isolation devices, or components, and the like.

The preceding description provides various embodiments of the spacer fluids containing different additives and concentrations thereof, as well as methods of using the spacer fluids. It should be understood that, although individual embodiments may be discussed herein, the present disclosure covers all combinations of the disclosed embodiments, including, without limitation, the different additive combinations, additive concentrations, and fluid properties.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present invention. If there is any conflict in the usages of a word or term in this specification and one or more patent(s) or other documents that may be incorporated herein by reference, the definitions that are consistent with this specification should be adopted.

What is claimed is:

1. A method of measuring a fluid's viscosity comprising:
flowing the fluid through a conduit wherein the conduit comprises shear stress sensors operable to measure shear stress on a wall of the conduit;
measuring shear stress using the shear stress sensors; and
calculating a viscosity of the fluid based at least in part on the measured shear stress.

2. The method of claim 1 wherein the shear stress sensors comprise a MEMS shear sensor.

3. The method of claim 1 wherein the step of calculating a viscosity comprises:
calculating rheological model parameters from a flow rate of the fluid and measured shear stress at the flow rate; and
calculating the viscosity based at least in part on a theological model that corresponds to the calculated rheological model parameters.

4. The method of claim 3 wherein the rheological model is a power law model in the form of:
τ=K\dot{y}ⁿ
where τ=shear stress and the rheological model parameters are K=viscosity constant, \dot{y}=shear rate, and n=power law exponent.

5. The method of claim 4 wherein the rheological model parameters are calculated based at least in part on the following equation:
$$Q = \frac{n π}{3 n + 1} {( \frac{{τ}_{w}}{K} )}^{\frac{1}{n}} {R}^{3 n}$$
where τ_{w} measured shear stress and R is radius.

6. The method of claim 4 wherein the conduit is a conical frustum with a variable diameter and wherein the rheological model parameters are calculated based at least in part on the following equation:
$$Q = \frac{n π}{3 n + 1} {( \frac{{τ}_{wi}}{K} )}^{\frac{1}{n}} {R}_{i}^{3 n}$$
where τ_{wi} is the shear stress measured at radius Ri.

7. The method of claim 3 wherein the rheological model is a Herschel-Bulkley model in the form of:
τ=τ₀+K\dot{y}ⁿ
where τ=shear stress, τ₀ is yield stress, and the rheological model parameters are K=viscosity constant, \dot{y}=shear rate, and n=power law exponent.

8. The method of claim 6 wherein the rheological model parameters are calculated based at least in part on the following equation:
$$Q = {( \frac{{τ}_{w} - {τ}_{0}}{K} )}^{\frac{1}{n}} \{ \frac{π {R}^{3}}{3} + \frac{{τ}_{w} π R}{K} * \frac{n}{n + 1} \} + \frac{{τ}_{w} π R}{K} * \frac{n}{n + 1} ( \frac{{τ}_{w} - {τ}_{0}}{K} ) + \frac{π {R}^{3}}{3} {( \frac{{τ}_{0}}{K} )}^{\frac{1}{n}}$$
where τ_{w} measured shear stress and R is radius.

9. The method of claim 3 wherein the rheological model parameters are calculated based at least in part on the following equation:
$$Q = {( \frac{{τ}_{wi} - {τ}_{0}}{K} )}^{\frac{1}{n}} \{ \frac{π {R}^{3}}{3} + \frac{{τ}_{wi} π {R}_{i}}{K} * \frac{n}{n + 1} \} + \frac{{τ}_{wi} π {R}_{i}}{K} * \frac{n}{n + 1} ( \frac{{τ}_{wi} - {τ}_{0}}{K} ) + \frac{π {R}_{i}^{3}}{3} {( \frac{{τ}_{0}}{K} )}^{\frac{1}{n}}$$
where τ_{wi} is the shear stress measured at radius Ri.

10. A method comprising:
circulating a drilling fluid through a wellbore penetrating a subterranean formation while drilling the wellbore;
flowing the fluid through a conduit wherein the conduit comprises shear stress sensors operable to measure shear stress on a wall of the conduit;
measuring shear stress using the shear stress sensors; and
calculating a viscosity of the fluid based at least in part on the measured shear stress.

11. The method of claim 10 wherein the shear stress sensors comprise a MEMS shear sensor.

12. The method of claim 10 wherein the step of calculating a viscosity comprises:
calculating rheological model parameters from a flow rate of the drilling fluid and measured shear stress at the flow rate; and
calculating the viscosity based at least in part on a rheological model that corresponds to the calculated theological model parameters.

13. The method of claim 12 wherein the rheological model is a power law model in the form of:
τ=K\dot{y}ⁿ
where τ=shear stress and the rheological model parameters are K=viscosity constant, \dot{y}=shear rate, and n=power law exponent, and wherein the rheological model parameters are calculated based at least in part on the following equation:
$$Q = \frac{n π}{3 n + 1} {( \frac{{τ}_{wi}}{K} )}^{\frac{1}{n}} {R}_{i}^{3 n}$$
where τ_{wi} is the shear stress measured at radius Ri.

14. The method of claim 12 wherein the conduit has a conical frustum geometry, wherein the rheological model is a Herschel-Bulkley model in the form of:
τ=τ₀+K\dot{y}ⁿ
where τ=shear stress, τ₀ is yield stress, and the rheological model parameters are K=viscosity constant, \dot{y}=shear rate, and n=power law exponent, and wherein the rheological model parameters are calculated based at least in part on the following equation:
$$Q = {( \frac{{τ}_{wi} - {τ}_{0}}{K} )}^{\frac{1}{n}} \{ \frac{π {R}^{3}}{3} + \frac{{τ}_{wi} π {R}_{i}}{K} * \frac{n}{n + 1} \} + \frac{{τ}_{wi} π {R}_{i}}{K} * \frac{n}{n + 1} ( \frac{{τ}_{wi} - {τ}_{0}}{K} ) + \frac{π {R}_{i}^{3}}{3} {( \frac{{τ}_{0}}{K} )}^{\frac{1}{n}}$$
where τ_{wi} is the shear stress measured at radius Ri.

15. The method of claim 10 further comprising:
comparing the viscosity of the drilling fluid to a setpoint viscosity;
calculating an amount of a chemical additive to add to the drilling fluid to reach the setpoint viscosity; and
adding a chemical additive to the drilling fluid based at least in part on the calculating.

16. The method of claim 15 wherein the chemical additive comprises at least one selected from the group consisting of a weighting agent, a viscosifier, a breaker, a base fluid, and combinations thereof.

17. A system comprising:
a line fluidly connecting a mixing tank and a tubular extending into a wellbore with a pump disposed along the line between the mixing tank and the tubular;
a conduit in fluid communication with the line between the mixing tank and the pump; and
one or more shear stress sensors disposed within the conduit operable to measure shear stress on a wall of the conduit.

18. The system of claim 17 further comprising at least one chemical additive tank coupled to the mixing tank.

19. The system of claim 18 further comprising a control system operable to calculate a viscosity of a fluid within the conduit, compare the viscosity to a setpoint viscosity, calculate an amount of a chemical additive to add to the mixing tank such that the viscosity of the fluid is adjusted to a value closer to the setpoint viscosity.

20. The system of claim 19 wherein the control system is configured to calculate viscosity by calculating rheological model parameters from a flow rate of the fluid through the conduit and measured shear stress at the flow rate; and calculate the viscosity based at least in part on a rheological model that corresponds to the calculated rheological model parameters.
